(19) United States
(12) Patent Application Publication
(10) Pub. No.: US 2010/0152786 A1
(43) Pub. Date: Jun. 17, 2010

(54) ORTHOPEDIC BONE FIXATION

(75) Inventor: Eyal Behrbalk, Even-Yehuda (IL)

Correspondence Address: MARTIN D. MOYNIHAN d/b/a PRTSI, INC., P.O. BOX 16446, ARLINGTON, VA, 22215, US

(73) Assignee: Ogen Innovative Medical Devices Ltd., Even-Yehuda (IL)

(21) Appl. No.: 12/442,994

(22) PCT Filed: Oct. 7, 2007
(86) PCT No.: PCT/IL2007/001197

§ 371 Date: Feb. 24, 2010

Related U.S. Application Data
(60) Provisional application No. 60/847,681, filed on Sep. 28, 2006.

(51) Int. Cl. A61B 17/86

(52) U.S. Cl. 606/301; 606/326

(57) ABSTRACT

The present invention provides a bone anchor, and a method of using the same, the bone anchor comprising a hollow cylindrical housing, buttress means attached to the distal end of said cylindrical housing comprising one or more releasable anchors, a threaded rod rotatably placed within the internal space of said housing, said threaded rod is mechanically linked to the buttress means, wherein said anchors are capable of being moved between an open conformation and a closed conformation and vice versa by means of manipulating the proximal end of said threaded rod.

### FIELD OF THE INVENTION

The present invention relates to an orthopedic fixation/anchoring device, and to a method of use thereof in a surgical procedure, for the repair of bone fractures and the fixation of orthopedic/dental implants. More particularly, the present invention relates to an orthopedic fixation/anchoring device, and to a method of use thereof, which permits carrying out bone fixation in a minimally invasive manner.

### BACKGROUND OF THE INVENTION

Improved medical care of the elderly has resulted in an increase in the mean age of the elderly who undergo orthopedic operations. In many orthopedic departments, the mean age of the patients is 85 years old. In is a goal of health care providers to quickly restore full ambulation to patients who suffer from bone fractures since the ability to move means life.

In a review concerning the fixation of osteoporotic fractures by Giannoudis et al (“Principles of fixation of osteoporotic fractures”, the journal of bone and joint surgery, Vol. 88-B, No. 10, October 2006, pp. 1272-1278), the various bone fixation treatment methods and techniques used nowadays are discussed. It is pointed out in this review that despite the significant developments in the field, surgeons are still facing many difficulties which are not addressed by the treatment means employed nowadays.

U.S. Pat. Nos. 5,893,850 and 6,348,053 describe a bone fixation device which utilizes an anchor provided in a form of axially-extending strips formed a long a tubular sleeve of the fixation device. This anchor is adapted to engage the cancellous (spongy) bone material by forcing the axially-extending strips to fold radially outwardly.

The orthopedic fastener described in U.S. Pat. No. 5,098,433 comprises a shaft having a pair of wings rotatably attached to its sides near its distal end, and a cylindrical tube capable of receiving the shaft and the wings attached thereto when aligned with an elongated axis of the device (closed state). The shaft and the wings attached thereto are inserted into a pre-drilled bore in the bone worked upon while enclosed in the cylindrical tube. The wings are opened by advancing the shaft in the tube to a point where the wings fully extend out of the upper end of the tube, in this state the wings are rotated radially away from the shaft by means of beveled sections formed on the upper face of the sleeve. Therefore, in order to achieve a distal anchor over the bone the shaft should be advanced distally out of the drilled bore such that the entire length of the wings is ejected via the remote opening of the bore.

Currently, the conventional devices utilized for bone fixation are mostly unable to fixate osteoporotic and low density bones in a solid, reliable manner. In many cases bone fractures are repaired by the use of screws which are held, if at all, loosely by the bone. There is therefore a need for improved fixation means for bone fixation treatments.

It is therefore an object of the present invention to provide an improved bone fixation device capable of distributing the load over a significant effective area in a given bone, thus reducing the burden (stress) on the bone and resulting in better fixation, improved stability, and significantly lower rate of hardware failure.

It is another object of the present invention to provide an improved bone fixation device, and a method for using the same, which provides improved rigid fixation of fractured bones.

It is yet another object of the present invention to provide an improved bone fixation device capable of acting as a platform on which other fixation devices could be anchored to, such as mooring of torn tendons to bones and spinal rods.

It is a further object of the present invention to provide an improved bone fixation which may be employed in conjunction with the conventional commonly used hardware, for example plates, such as locking plates and dynamic compression plates.

It is a yet a further object of the present invention to provide a “one size fits all” bone fixation device, and thereby to reduce the number of variants needed to cover a wide range of bone sizes.

It is another object of the present invention to provide a bone fixation device which may cover a wide range of fixation angles.

It is yet another object of the present invention to provide a bone fixation device, and method of use thereof, which allows easily retrieving the fixation device by means of a reciprocal procedure.

It is yet another object of the present invention to provide a bone fixation device capable of introducing and releasing drugs such as antibiotics, growth factors, and bone morphogenic proteins.

Other objects and advantages of the invention will become apparent as the description proceeds.

### SUMMARY OF THE INVENTION

The present invention is directed to a bone anchor comprising a cylindrical housing and a buttress mechanism operable to release one or more anchoring means movably attached therein, said bone anchor is adapted to be inserted into a channel drilled in the bone worked upon and deploy the anchors by means of a threaded rod rotatably mounted in the cylindrical housing and mechanically linked to the buttress mechanism.

The anchoring means are released via lateral apertures provided in a distal portion of the cylindrical housing which may further comprise curved passages connecting between the lateral apertures and its internal space. The anchoring means may be released via the lateral aperture substantially perpendicular to the cylindrical housing. Alternatively, the lateral apertures and/or the curved passages are configured to release the anchoring means such that the released anchoring means are slanted proximally, or optionally curled proximally.

After distal anchor is obtained by means of the releasable anchors, a proximal anchor is preferably applied by means of a nut mounted on a threaded proximal portion formed on the external surface of the cylindrical housing, or by means of a bolt employing an internal thread provided in a proximal end portion of the cylindrical housing.

The anchoring means of the invention may further comprise a gripping element capable of receiving a distal portion of at least one anchoring means in its released state in a side opening thereof, and clamping said distal portion by clamping means provided thereinside. The clamping means may be implemented by a threaded rod capable of being threaded in a threaded channel provided in the gripping element. In this way, an improved fixation of a bone may be achieved by passing the bone anchor of the invention and the gripping element in adjacent channels drilled in the bone, releasing the anchoring means such that a distal portion of at least one of the anchoring means is received in the side opening of the gripping element, fastening the clamping mechanism over the distal portion of the at least one anchoring means, and proximally anchoring the bone anchor and the gripping element by means of nuts adapted to be threaded over their proximal portions. The fixation may be further improved by rotating the threaded rod of the bone anchor such that portions of the released anchoring means are retracted proximally.

The term proximal (near) used herein to refer to parts of the bone or of the bone fixation device which are located adjacent to the surgeon or to the surgical incision.

The term distal (far) used herein to refer to parts of the bone or of the bone fixation device which are in a remote location relative to the surgeon or to the surgical incision.

The term cancelous bone used herein refers to spongy bone, trabecular bone, e.g. can be found in the metaphysial part of long bones.

The term cortical bone used to refer to compact bone, high density bone, e.g. the bone layer enveloping the diaphysis of long bones.

The term buttress mechanism used herein to refer to a mechanism designed for applying a distal anchor over, or inside, a bone by means of movable anchoring means.

In one aspect the present invention is directed to a bone anchor comprising:
(a) a hollow cylindrical housing;
(b) buttress means attached to the distal end of said cylindrical housing comprising one or more releasable anchors;
(c) a threaded rod rotatably placed within the internal space of said housing, said threaded rod is mechanically linked to the buttress means;
wherein said anchors are capable of being moved between an open conformation and a closed conformation and vice versa by means of manipulating the proximal end of said threaded rod.

The buttress means of the bone anchor preferably comprises a threaded bore adapted to thread over the threaded rod such that said buttress means may be advanced distally, inside the hollow cylindrical housing, or retracted proximally, responsive to rotations of said threaded rod.

Advantageously, the anchors are capable of being moved from an open conformation to a closed conformation by means of rotating the threaded rod in a first direction, and said anchors are also capable of being moved from a closed conformation to an open conformation by means of rotating said threaded rod in a second direction.

Advantageously, after releasing the anchors a proximal section of the bone anchor is secured to the bone by means of a nut or a bolt.

Preferably, the anchor is suitable for insertion into a bone surface at any desired angle to said surface.

A distal portion of the hollow cylindrical housing may comprise lateral apertures, wherein said lateral apertures are adapted to provide a passage to portions of the releasable anchors, and wherein the lateral apertures are configured to eject the portions of the releasable anchors in a predetermined angle relative to the longitudinal axis of the hollow cylindrical housing. Additionally, the hollow cylindrical housing may comprise one or more passages connecting between its internal space and the lateral apertures, and wherein said passages are configured to eject the portions of the releasable anchors in a predetermined angle relative to the longitudinal axis of the hollow cylindrical housing.

The bone anchor may be used in combination with a gripping means capable of receiving and clamping a distal portion of the releasable anchors when in their opened state, wherein said gripping means comprises a hollow cylindrical housing having a distal side opening and clamping means.

In another aspect the present invention is directed to a surgical procedure for bone fixation comprising the steps of:
i) drilling a channel in the bone to be fixated from the proximal cortex to the distal cortex;
ii) inserting a bone anchor into the drilled channel through the proximal opening thereof until the distal edge of said anchor exits the bone outside the distal cortex through the distal opening of said channel;
iii) manipulating the device to release one or more anchors outside the distal cortex; and
iv) pulling on the proximal end of the bone anchor so that the released support buttress rests against the external surface of the distal cortex of the bone.

Alternatively, the bone anchor may be used to provide an internal anchor over the proximal cortex by positioning said bone anchor inside the drilled bore such that the one or more anchors are released beyond the proximal cortex—namely, inside the bone. In this case the drilled bore passes only the proximal cortex.

Conveniently, the manipulation of the device in order to release one or more anchors comprises rotating the proximal end of a threaded rod. Optionally, the rotation of the proximal end of the threaded rod is achieved using a dedicated tool having a distal end shaped to correspond to a recess in the proximal end of said rod.

Advantageously, the length of the cylindrical housing is greater than the diameter of the bone for permitting proximal cleaving it by means of a nut (bolt mechanism) adapted to thread over the proximal section of the cylindrical housing. In this way, a “one size fits all” bone fixation device may be achieved. Alternatively, if a bolt mechanism is employed for achieving the proximal cleaving by means of a proximal section of the threaded rod than the length of the elongated cylindrical housing may be shorter than the diameter of the bone.

After achieving proximal anchor by means of the nut/bolt mechanism the residual of the elongated cylindrical housing (the portion thereof protruding proximally from the bone) may be trimmed by means of suitable trimming instrument. The proximal section of the elongated cylindrical housing may comprise shearing grooves formed therealong for facilitating the trimming.

### BRIEF DESCRIPTION OF THE DRAWINGS

The present invention is illustrated by way of example in the accompanying drawings, in which similar references consistently indicate similar elements.

FIGS. 1A to 1C shows perspective views of an embodiment in which the support buttress is implemented by support pins (also referred to herein as wings), wherein FIG. 1A shows the fully assembled device, FIG. 1B shows the device without the cylindrical hollow housing, and FIG. 1C shows the fully assembled device without the distal cover of the cylindrical hollow housing;

FIG. 2 shows a perspective view of an embodiment of the invention attached to a tooling device (used for the proximal cleaving by means of a nut/bolt) and a centrally placed rod (for opening and closing the wings);

FIGS. 3A to 3D illustrate the steps of a bone fixation procedure utilizing the fixation device of the invention;

FIGS. 4A to 4M schematically illustrate the components and various operation states of an embodiment of the invention wherein the support buttress mechanism is implemented by a bendable strip;

FIGS. 5A and 5E illustrate an embodiment of the device shown in FIGS. 4A to 4M wherein the bendable strip is adapted to curl proximally when released;

FIGS. 6A to 6G illustrate embodiments of the device shown in FIGS. 4A to 4M comprising two bendable strips;

FIGS. 7A and 7B illustrate embodiments of the device shown in FIGS. 4A to 4M comprising two bendable strips which are adapted to curl proximally; and

FIGS. 8A to 8C illustrate an embodiment of the invention wherein a gripping element is used in combination with the device shown in FIGS. 4A to 4M for providing a distal grip of the anchoring means.

It should be noted that the embodiments exemplified in the Figs. are not intended to be in scale and are in diagram form to facilitate ease of understanding and description. The number of wings and their curving angles can be changed to fit different needs of the surgeon.

### DETAILED DESCRIPTION OF PREFERRED EMBODIMENTS

The present invention provides an improved bone fixation device especially suitable for treatment of bone fractures and to serve as an implant device on which other hardware can be anchored to or by it. The bone fixation device of the present invention comprises a support buttress mechanism at its distal end, designed to provide an anchor over a remote opening of a bore drilled in the bone. The anchoring can be achieved by means of a standalone unit having a single wing or two wings (i.e., anchoring means), said wing(s) may be applied in a substantially vertical, or entangled form, relative to the bone fixation device. The bone fixation device may be used in combination with a gripping element adapted to grip the anchoring wing(s) of the bone fixation device thereby forming a “bridge” over a distal portion of the bone (either over the far cortex or inside the bone over the near cortex). The wings can be retracted proximally, thereby providing a safe extraction of the apparatus.

The fixation device of the invention comprises a hollow cylindrical housing adapted to receive a support buttress mechanism in a distal portion of its interior, said hollow cylindrical housing comprises threading provided on its proximal section capable of receiving a nut/bolt. The nut is used for applying a cleaving force between the proximal end of the shaft and the anchor provided by the anchoring means of the support buttress mechanism when released over the distal side of the device. The nut (or bolt) is deployed only after the anchoring means are released over the proximal cortex, inside the bone, or over the distal cortex outside the bone, thereby applying the cleaving forces needed for the secure fixation.

As will be described herein in details the anchoring means are laterally ejected from the fixation device by rotating a threaded rod attached inside the device, said threaded rod is mechanically linked to the support buttressing mechanism.

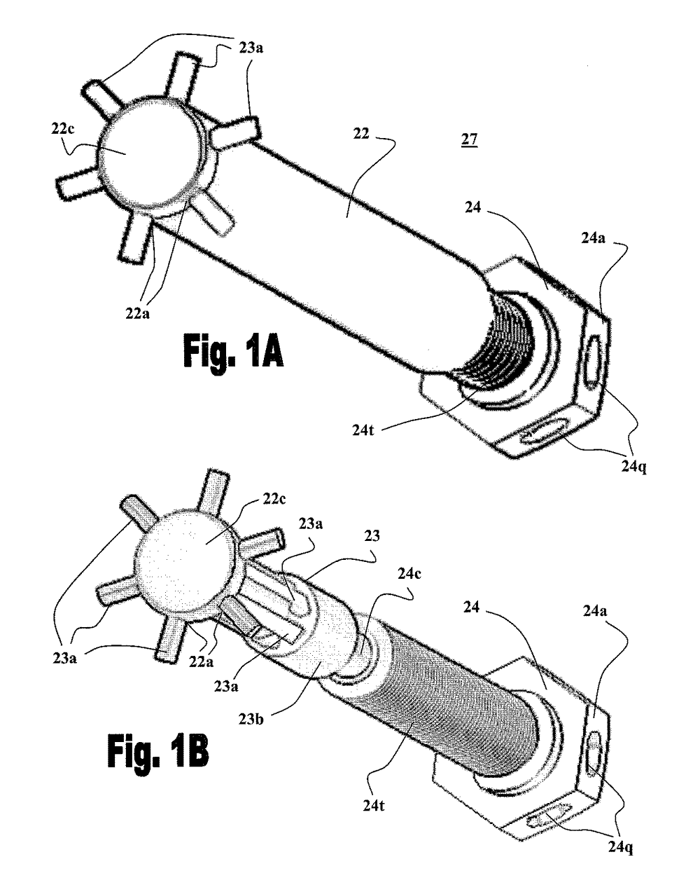

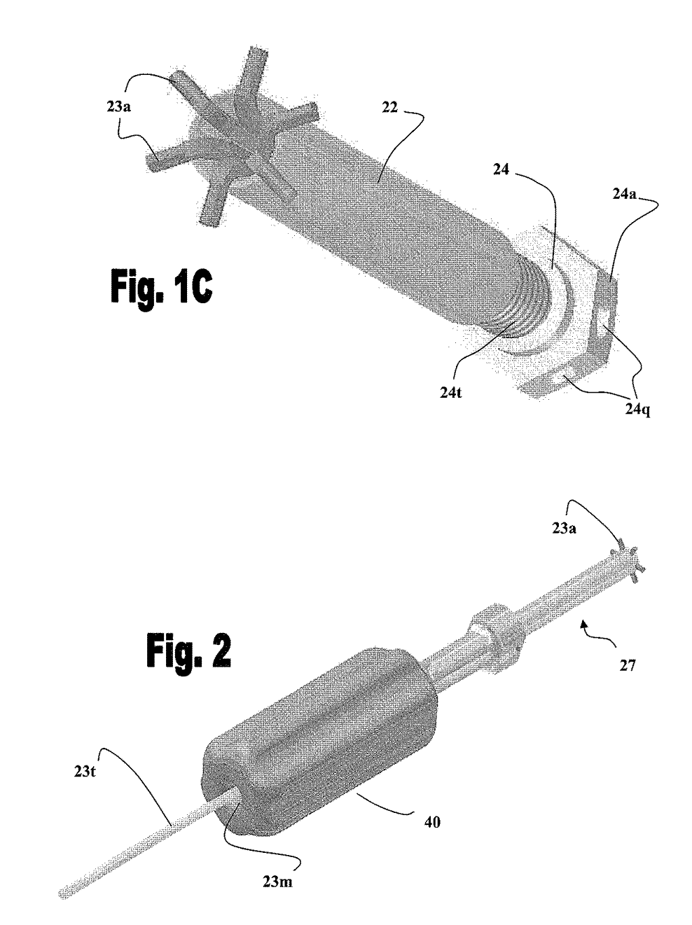

FIG. 1A to 1C shows perspective views of a fixation device 27 of the invention in which the support buttress mechanism 23 is implemented by support pins 23a. With reference to FIG. 1A, fixation device 27 comprises a cylindrical hollow housing 22 having circumferential lateral apertures 22a formed on a distal portion thereof, a bolt 24 comprising a threaded shaft 24t having a bolt head 24a at its proximal end, and support buttress mechanism 23 (shown in FIG. 1B) installed inside cylindrical hollow housing 22. A proximal portion of the inner wall of cylindrical hollow housing 22 comprises threads (not shown) corresponding to the threads of threaded shaft 24t to allow progressively advancing/retracting bolt 24 into/from the interior of cylindrical hollow housing 22 by rotating it about its axis. Bolt head 24a of bolt 24 may comprise radial bores 24q for facilitating the griping of the fixation apparatus by the tooling device (40, shown in FIG. 2).

As seen in FIG. 1B, wherein fixation device 27 is shown without cylindrical hollow housing 22, the distal end of bolt comprises a centrally positioned rod 24c mechanically linked, to base 23b of support buttress mechanism 23, and adapted to advance/retract base 23b in response to axial movements of bolt 24. The proximal end of support pins 23a is attached to base 23b such that distal end portions of support pins 23a are forced to move laterally in, or out, via circumferential lateral apertures 22a of cylindrical hollow housing 22 in response to axial movements of rod 24c. FIG. 10 shows fixation device 27 without the distal cover 22c of the cylindrical hollow housing 22.

In a preferred embodiment of the invention distal cover 22c is an integral part of cylindrical hollow housing 22 and it preferably provides guiding tunnels for the support pins 23a, said guiding tunnels are configured to bend support pins 23a when they are laterally ejected therefrom.

Initially, the entire length of the support pins (wings) 23a is within fixation device 27. In this state fixation device 27 can be smoothly advanced through a bore drilled in the treated bone. Thereafter, anchor over the remote (distal) opening of the drilled bore is established by rotating rode 24c to progressively advance it into cylindrical hollow housing 22, and thereby force distal end portion of support pins (wings) 23a to eject laterally via circumferential lateral apertures 22a of cylindrical hollow housing 22. Finally, a proximal anchor is established by tightening bolt 24 over the proximal side of the bone (or over a fixating plate attached thereto) by threading it axially over the cylindrical housing 24.

Similarly, fixation device 27 may be removed from the drilled bore by unfastening the proximal bolt head 24a, than retracting rod 24c proximally to progressively retrieve support pins 23a into cylindrical hollow housing 22 via its circumferential lateral apertures 22a.

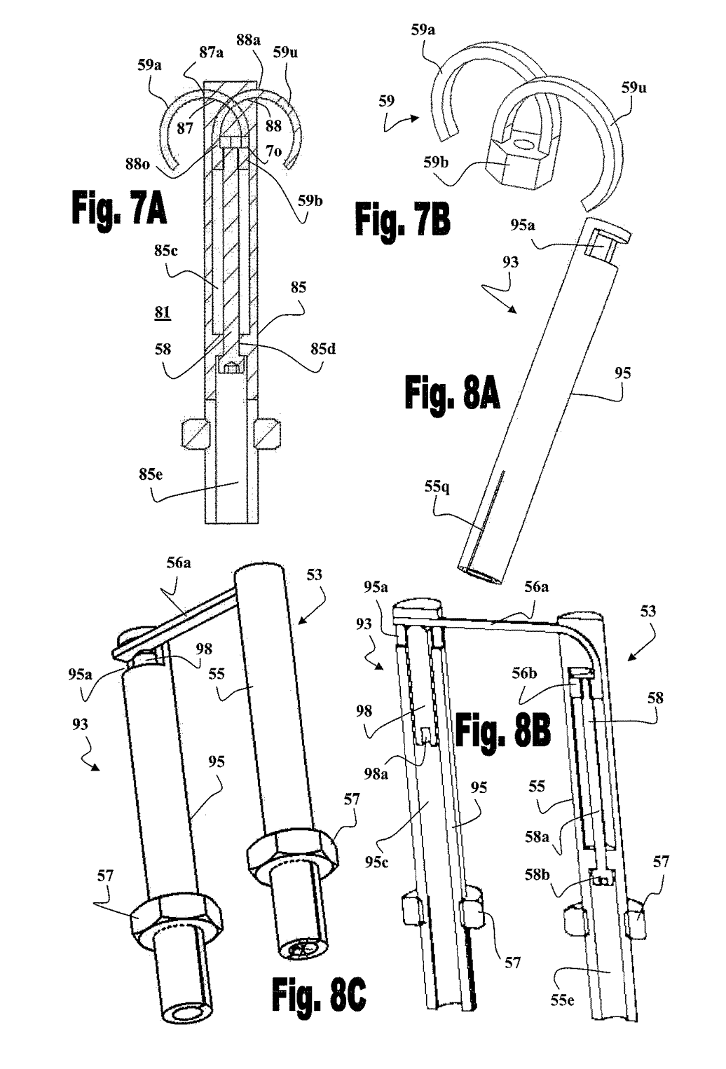

Cylindrical hollow housing 22 and bolt 24 may be manufactured from types of long term implant approved materials such as, but not limited to, titanium, medical grade stainless-steel, composite materials, plastic. The diameter of cylindrical hollow housing 22 may generally be in the range of 3 to 5 mm. In general, the length of cylindrical housing 22 should be shorter than the diameter of the bone, if a screw/bolt is used to fasten the proximal side of the fixation device or, longer than the diameter of the bone if a nut is used to fasten the proximal side of the device. Support pins 23a are preferably made from a type of long term implant approved material, such as indicated above. The length of the laterally ejected portion of support pins 23a may be in the range of 3 to 30 mm, depending on the situation in which the device is deployed. For example, if the device is a standalone device (i.e., a gripping element is not used) a 3 mm length may suffice, albeit, if the device is used in combination with a gripping element (described herein later with reference to FIGS. 8A-8C) the support pins are deployed to a length of about 30 mm. The diameter of support pins may generally be in the range of 1 to 3 mm.

Bolt 24 and bolt head 24a may be manufactured from a type of long term approved implant material, such as indicated above. The diameter of shaft 24t of bolt 24 should be adapted to be inserted into cylindrical housing 22, and its length may generally be in the range of 5 to 30 mm.

The parts of the device may be manufactured and processed in various methods, such as, but not limited to, Molding, Lathing, sintering, Laser Cutting, Forging and thermal treatment. Internal/external surfaces of the bone fixation device may be covered by a coating specially adapted to reduce friction. For example, Teflon coating, such coating will advantageously reduce the friction between movable members of the device, and will also prevent attachment of the healing bone to the device. Alternatively or additionally, the internal/external surfaces of the bone fixation device may be covered by an antibiotic coating for preventing infection.

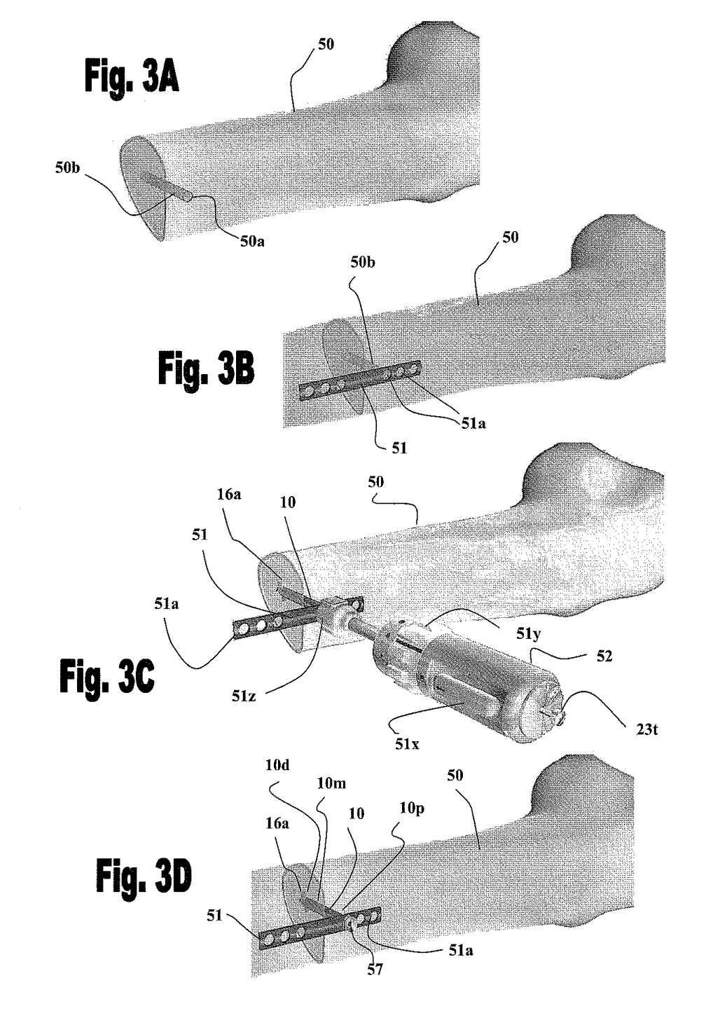

As illustrated in FIG. 3D, after placement of the fixation device 10 its proximal part 10p forms the buttress on the proximal side of the bone 50, its middle part 10m i.e., the cylindrical housing of the device, is inside bone 50, and its distal part 10d forms the support buttress on the distal cortex, or inside the bone in cancellous bone, or on the proximal cortex in a cortical bone, thus creating the cleaving force to fix and stabilize bone fractures and thereby improve primary healing.

The proximal part 10p is formed by a bolt 24 and bolt head 24a that is fastened over the bone, or a fixation plate attached thereto. In one embodiment, the bolt 24 has a central hole (not shown) passing along the length of bolt 24, said central hole is adapted to receive a rod 23t which is utilized to deploy or retrieve the support pins 23a.

The middle part i.e., cylindrical housing 22, comprises a threading formed on a proximal section of its internal wall, said threading holds bolt 24. On its distal end rod 24c comprises means adapted to hold the support buttress mechanism, which push and eject, and thereby form the buttress, the anchoring means over the distal side of the bone. The middle part 22 may be shorter than the diameter of the bone, if the proximal cleaving is achieved by means of bolt 24 adapted to engage threading in the cylindrical housing. Alternatively, the middle part 22 is made longer than the diameter of the bone when the proximal cleaving is achieved by means of a nut adapted to thread over a proximal portion of the middle part 22, thus allowing the fixation device of the invention to fit to a wide range of bone diameters. A main advantage of the bone fixation device of the invention over known fixation screws of the art is that the surgeon will need only one size device in order to fit to the bone 50 diameter.

The distal part 10d of the fixation device attaches to the middle part 10m and includes the support buttress and its mechanism 23, which comprises one or more releasable anchoring means (wings) 23a which may be deployed (or retrieved) over the distal side of the bone. As illustrated in FIG. 1A, the support buttress may be implemented in wide variety of angles relative to the longitudinal axis of the device, thus, if needed, the fixation device of the invention can be inserted into the bone in various insertion angles, for example, offering the possibility to insert the device orthogonal to the fracture line (not shown).

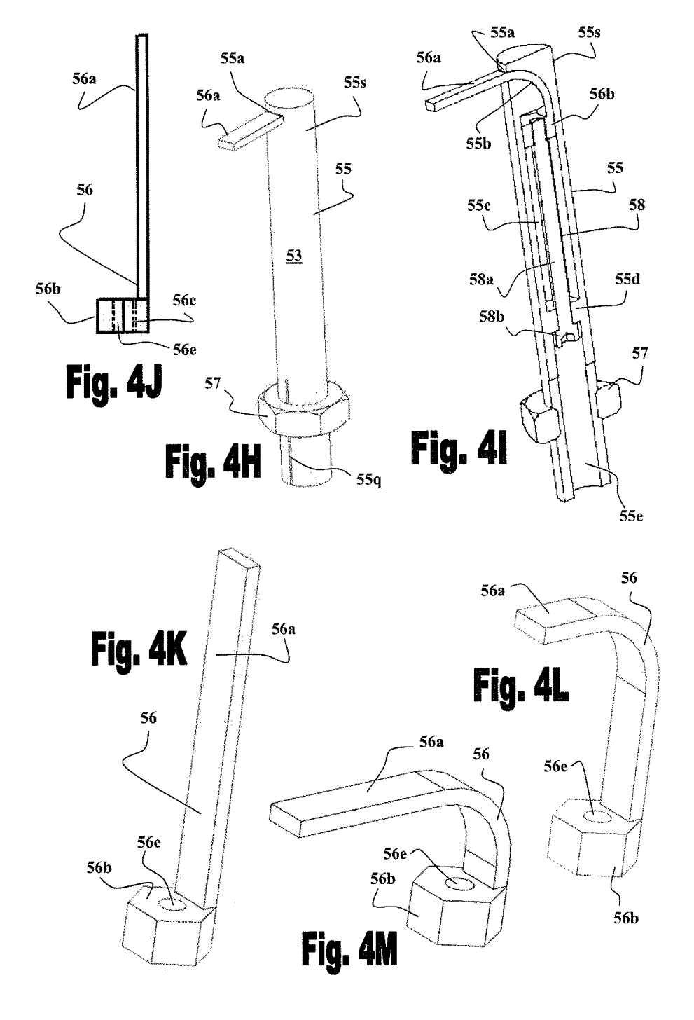

In one specific embodiment of the invention the fixation device further comprises a central passage 23m, passing along the tooling device 40, along bolt 24 head, and all the way to the buttressing mechanism base 23b. A hexagonal rod 23t, shown in FIGS. 2 and 5C, is inserted through central passage 23m in order to deploy or retrieve the supporting wings buttress at the far side of the bone.

FIG. 2 shows a perspective view of fixation device 27 attached to a tooling device 40 and having a rod 23t passing thereinside for manipulating the buttress as described above.

FIGS. 3A to 3D illustrate use of an embodiment of the fixation device 10 of the invention in a bone fixation procedure. The bone fixation procedure starts after the surgeon exposes the fractured area, and reduces the fracture to an acceptable position. Bone fixation can be performed either by the device itself, or by using it with commonly used hardware as for example fixation plates (51), which bridges across the fracture. In this example, the fixation device of the invention fastens and secures the plate to the bone.

The procedure starts by drilling a 3-5 mm diameter bore 50b in a treated bone 50 in a way that an aperture 51a of fixation plate 51 communicates with the proximal opening 50a of the drilled bore 50b. Then fixation device 10 is inserted into bore 50b and the releasable anchoring means (wings) 16a are deployed by support buttress mechanism of fixation device 10 by means of tooling device 52. After distal anchoring is achieved by the releasable anchoring means 16a, a proximal anchoring is obtained by tightening a bolt/nut 57 (or 24) over the proximal end section of the cylindrical hollow housing (22 or 55) of the fixation device. In order to facilitate the proximal anchoring of the cylindrical hollow housing a thread suitable for receiving bolt/nut 57 (or 24) is engraved on a proximal portion of the outer/inner wall of cylindrical hollow housing 22.

Rod 23t serves to deploy or retrieve the anchoring means of the support buttress mechanism. Rod 23t traverses through the central passage in the tooling device center 52 (or 40). The tooling device 52 fits onto the nut 57 and locks it firmly. Rotating the tooling device in one direction e.g., clockwise, rotates the nut 57 and thereby causes the nut to axially advance onto/into the cylindrical housing 22. In this way the fixation device is secured to the bone firmly, by forming cleaving forces with the distal anchoring means wings. Rotating the tooling device in the other direction e.g., counter clockwise reverses the above mentioned process and allows for the retrieval/extraction of the apparatus.

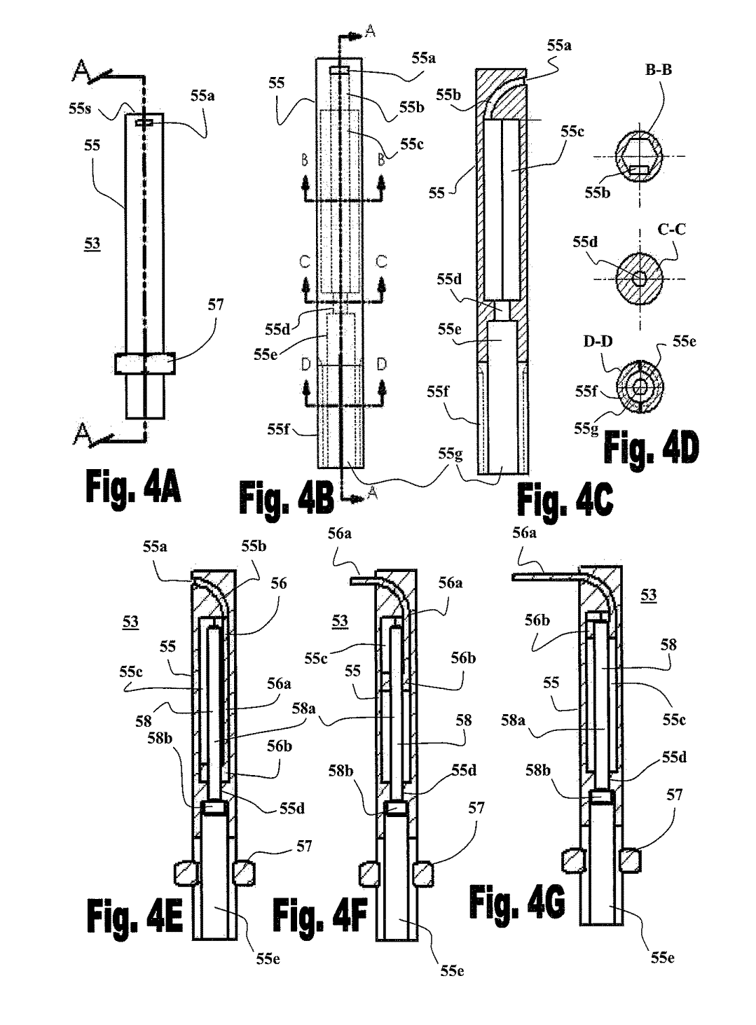

FIGS. 4A to 4M schematically illustrate the components and various operation states of yet another variation of a fixation device 53 of the invention. FIG. 4A shows a side view of fixation device 53 comprising a hollow cylindrical housing 55 having a lateral aperture 55a near its distal end 55s and a nut 57 threaded over threads 55f (shown in FIG. 4B) provided over a proximal end portion of the hollow cylindrical housing 55.

FIG. 4B illustrates a side view of the hollow cylindrical housing 55, showing its internal parts in dotted lines. Cylindrical hollow housing 55 comprises of a proximal cavity 55e and a distal cavity 55c. Said cavities are connected by a slender passage 55d. Proximal cavity 55e may be accessed via a proximal opening 55g. Curved passage 55b provided in hollow cylindrical housing 55 is used for communicating between distal cavity 55c and lateral aperture 55a.

FIG. 4C shows a longitudinal cross section view of the hollow cylindrical housing 55 taken along line A-A.

Distal cavity 55c is adapted to include anchoring means 56 mechanically linked to central bolt 58, such that the anchoring means 56 may be pushed distally through the lateral aperture 55a by rotating central bolt 58.

FIG. 4J illustrates a side view of releasable anchoring means 56. Releasable anchoring means 56 comprises a base 56b and a flexible strip 56a. One end of flexible strip 56a is preferably attached to base 56b near one of its lateral sides, such that a 90° angle is formed between base 56b and flexible strip 56a. Base 56b comprises a central bore 56e passing along its axis, said central bore 56e includes a thread 56c. As seen in the perspective view shown in FIG. 4K, base 56b may have a polygonal geometry (e.g. triangle, hexagon).

FIG. 4D shows cross-sectional views of the hollow cylindrical housing 55 taken along lines B-B, C-C and D-D. As seen in cross-sectional view B-B cross sectional geometry of distal cavity 55c may favorably be of polygonal shape (e.g., hexagon) to permit anchoring means 56 to slide therealong while preventing rotations thereof. As illustrated in cross-sectional view D-D shown in FIG. 4D, the diameter of proximal opening 55g may be reduced relative to the diameter of near cavity 55e such that the rod 23t may easily passed therethrough and directed towards the head 58b of bolt 58 mounted in slender passage 55d (shown in FIG. 4B).

With reference to FIGS. 4E, 4F and 4G, showing longitudinal-section views of fixation device 53 in different states of deployment, fixation device 53 further comprises a bolt 58 comprising a head 58b and a treaded rod 58a. Bolt 58 is mounted in slender passage 55d such that its head 58b is rotatably placed in a distal section of proximal cavity 55e and its threaded rod 58a is mostly placed inside the distal cavity 55c. Slidable anchoring means 56 is placed inside distal cavity 55c and threaded rod 58a is passed through its central bore 56e passing through base 56b, such that threads 56c provided in central bore 56e are engaged with the threads of threaded rod 58a, thereby allowing to progressively advance/retract slidable anchoring means 56 in response to rotations of bolt 58.

FIG. 4E illustrates a longitudinal cross section view of fixation device 53 before deploying the anchoring means 56. In this state base 56b of slidable anchoring means 56 is placed near the proximal end of distal cavity 55c and the entire length of its bendable strip 56a is housed inside distal cavity 55c. Of course, a distal end portion of bendable strip 56a may be housed in curved passage 55b in this state.

FIG. 4F illustrates a longitudinal-section view of fixation device 53 during the application of the anchoring means 56. At this intermediate state, slidable anchoring means 56 is advanced by rotating bolt 58 such that a distal portion of bendable strip 56a is forced to move through curved passage 55b and a distal end section thereof laterally protrudes via lateral aperture 55a of cylindrical hollow housing 55. As shown in FIG. 4F, a portion of bendable strip 56a housed in curved passage 55b is forced to assume the curvature of curved passage 55b.

FIG. 4G illustrates a longitudinal cross section view of fixation device 53 after anchoring means 56 is deployed. At this state, slidable anchoring means 56 is advanced by rotations of bolt 58 such that its base 56b reaches, or is very close to, the distal end of threaded rod 58a of bolt 58. As seen in FIG. 4G, at this state a significant length of bendable strip 56a is laterally ejected via lateral aperture 55a. In this preferred embodiment curved passage 55b and lateral aperture 55a are configure such that the distal portion of bendable strip 56a protruding via lateral aperture 55a is orthogonal to the longitudinal axis of fixation device 53.

A perspective view of device 53 is shown in FIG. 4H, wherein bendable strip 56a of the anchoring means (56) is laterally ejected via lateral aperture 55a. FIG. 4I shows a perspective longitudinal-section view of device 53 in the same state as in FIG. 4H. FIGS. 4K, 4L and 4M, respectively show anchoring means 56 in its initial state (straight), in an intermediate state (partially ejected) and in a fully ejected state.

In fractured bone fixation procedure, fixation device 53 is used in a similar way as described hereinabove with reference to the previous embodiments of the invention. After drilling a bore in the bone, fixation device 53 is inserted into the bore such that a distal end portion thereof comprising lateral aperture 55a is protruding out of the bore via its remote (distal) opening. At this state slidable anchoring means 56 is advanced until a significant length of bendable strip 56a laterally protrudes via lateral aperture 55a and the desired distal anchoring is achieved. Thereafter, nut 57 is tightened to establish a proximal anchor of fixation device 53 over the bone, or over a fixation plate mounted thereon.

Cylindrical hollow housing 55 may be manufactured from a type of long term implant approved material, for example, medical stainless steel 316, titanium alloy, nickel chrome alloy, or composite materials, by means of Molding, Lathing, Sintering, Laser Cutting, Forging.

The length of cylindrical hollow housing 55 may, generally, be in the range of 15 to 60 mm, and its diameter may generally be in the range of 3 to 5 mm. The length of distal cavity 55c may, generally, be in the range of 4 to 30 mm, and its diameter may generally by in the range of 2.5 to 4 mm. The length of near cavity 55e may, generally, be in the range of 10 to 35 mm, and its diameter may, generally, be in the range of 2.5 to 4 mm.

Anchoring means 56 may be manufactured from a type of long term implant approved material, for example, medical stainless steel 316, titanium alloy, nickel chrome alloy, or composite materials. The length of bendable strip 56a of anchoring means may generally be in the range of 3 to 30 mm, and its thickness may generally be in the range of 0.5 to 1.5 mm.

FIGS. 5A to 5E illustrate an embodiment of the fixation device 53 shown in FIGS. 4A to 4M, wherein the bendable strip 56a is adapted to curl proximally when ejected via lateral aperture 55a. This proximal curve may be achieved by altering the geometry of the curved passage 55b, which allows ejecting bendable strip 56a into different angles. FIGS. 5A and 5B show longitudinal section views of device 53 in an intermediate state and in a fully ejected state, of anchoring means 56. FIG. 5C shows a perspective longitudinal-section view of device 53 showing anchoring means 56 in the fully ejected state. FIGS. 5D and 5E respectively show perspective views of anchoring means 56 in an intermediate state and in a fully ejected state

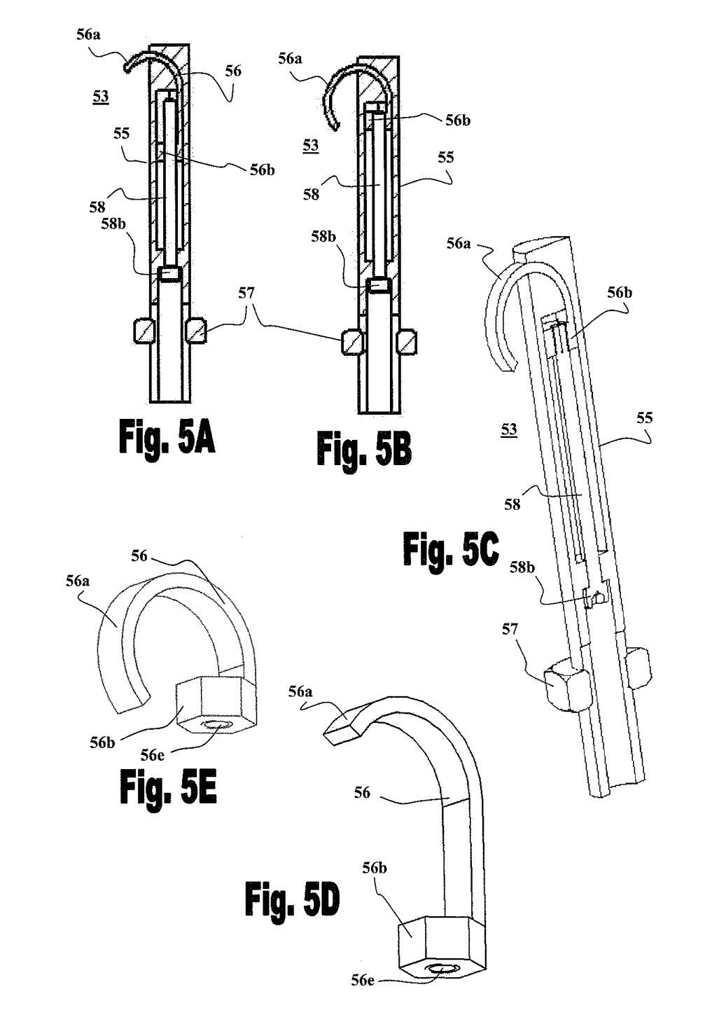

FIGS. 6A to 6F illustrate embodiments of a fixation device 80 of the invention comprising two bendable strips 59a and 59u. Fixation device 80 is substantially similar in structure and principle of operation to fixation device 53 shown in FIGS. 5A to 4M.

FIG. 6A illustrates a front view of slidable anchoring means 59 comprising a first bendable strip 59a and a second bendable strip 59u and a base 59b. The proximal ends of first bendable strip 59a and second bendable strip 59u are attached to opposing sides of base 59b.

FIG. 6B illustrates a top view of slidable anchoring means 59. As best seen in the perspective view of anchoring means 59 shown FIG. 6C, base 59b may have a polygonal geometry, for example hexagonal, and the first bendable strip 59a and second bendable strip 59u are preferably attached adjacent to two opposing corners thereof. Base 59b comprises a central bore 59e having threads 59c adapted to be engaged with thread of bolt 58 rotatably mounted in cylindrical hollow housing 85 (FIG. 6F).

With reference to the longitudinal section views of device 80 shown in FIGS. 6F and 6G, cylindrical hollow housing 85 comprises a proximal cavity 85e, a distal cavity 85c which communicates with proximal cavity 85e via a slender passage 85d, and two curved passages, 87 and 88, formed in two opposing sides of cylindrical hollow housing 85. Cylindrical hollow housing 85 further comprises two lateral apertures 87a and 88a, wherein lateral aperture 87a is located in the side of cylindrical hollow housing 85 opposing the side of opening 87o of curved passage 87, and lateral aperture 88a is located in the side of cylindrical hollow housing 85 opposing the side of opening 88o of curved passage 88.

As shown in FIGS. 6F and 6G, fixation device 80 establishes a distal anchor by means of two bendable strips 59a and 59u, adapted to laterally eject via respective apertures 87a and 88a, wherein the ejected portions of said strips are orthogonal relative to the longitudinal axis of fixation device 80. As described in the previous embodiment (fixation device 53) the bendable strips are progressively advanced by rotations of bolt 58. FIGS. 6C to 6E respectively show perspective views of anchoring means in its initial state (straight), intermediate state and in a fully ejected state.

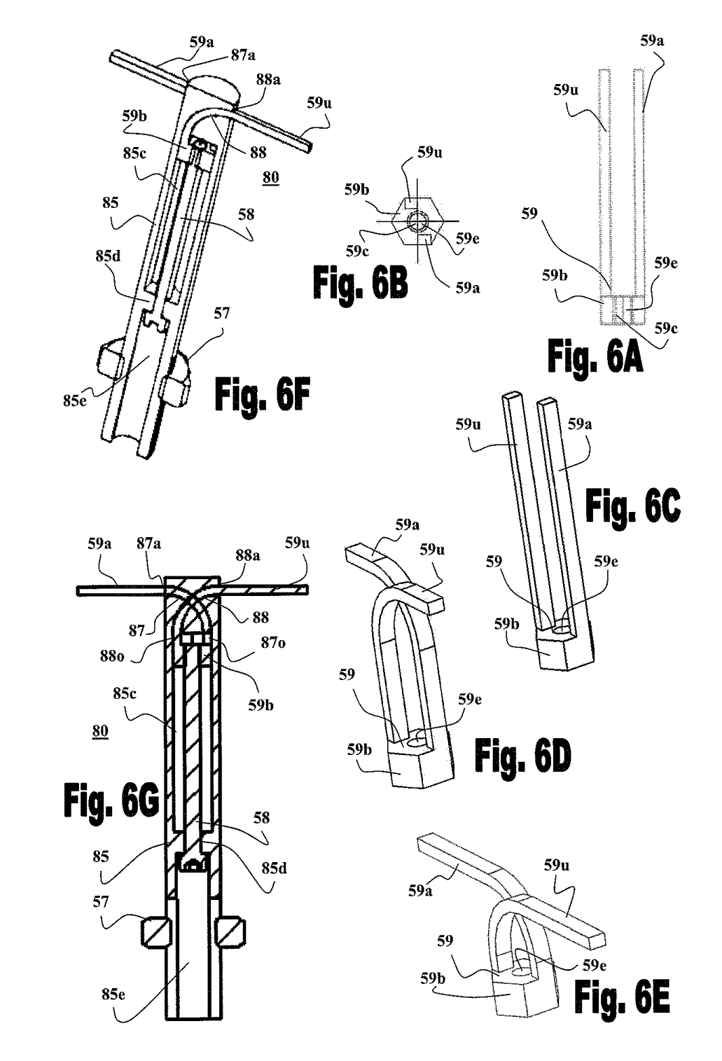

FIGS. 7A and 7B respectfully show a longitudinal-section view of a similar fixation device 81, and a perspective view of its anchoring means 59 in a fully ejected state. Fixation device 81 comprises a pair of bendable strips 59a and 59u which are adapted to curl proximally when ejected via respective apertures 87a and 88a.

FIGS. 8A to 8C illustrate a further embodiment of the invention which utilizes a gripping element 93 to achieve a distal grip of the ejected anchoring means 56. FIG. 8A shows a perspective view of gripping element 93 comprising a cylindrical hollow housing 95 and a distal side opening 95a. As shown in the perspective views shown in FIGS. 8B and 8C, distal side opening 95a is adapted to receive a distal end portion of bendable strip 56a in its ejected state. Said distal end portion is then clamped by a threaded bolt 98 which is threaded in threading formed in the internal wall of cavity 95c of cylindrical housing 95 until its distal end is firmly pressed against the distal end portion of bendable strip 56a received in distal side opening 95a. Gripping element 93 further comprises a nut 57 adapted to thread over threading provided on the proximal section of cylindrical housing 95 for anchoring it over a proximal portion of the bone.

In a bone fixation procedure gripping element 93 and fixation device 53 are introduced into adjacent bores (not shown) drilled in a treated bone such that their distal ends protrudes via the distal openings of the drilled bores. Gripping element 93 is placed such that the distal end of bendable strip 56a is received in its side opening 95a. After ejecting bendable strip 56a by means of bolt 58 such that a distal end portion thereof is received in side opening 95a, threaded bolt 98 is threaded distally (e.g., by means of proximal slit 98a) until its distal tip is firmly presses against the distal end portion of bendable strip. In this state, wherein gripping element 93 grips bendable strip 56a, nuts 57 of gripping element 93 and of fixation device 53 may be tightened over the bone (or over a fixation plate attached thereon), thereby firmly fixating the bone.

Gripping element 93 may be manufactured from a type of long term implant approved material, for example, medical stainless steel 316, titanium alloy, nickel chrome alloy, or composite materials. The length of gripping element 93 may generally be in the range of 15 to 60 mm, and its thickness may generally be in the range of 3 to 6 mm.

In one specific embodiment of the present invention, the distal buttressing mechanism of the fixation device is made from a type of elastic material, approved for long term implant by the FDA, such as silicon, rubber, plastic, nitinol capable of providing the needed distal anchor by manipulating its geometry. In this way, once the buttressing mechanism of the fixation device is passed (i) through the distal bone cortex via the distal opening of the drilled bore, or (ii) through the proximal cortex, via the proximal opening of the drilled bore, or (iii) into a cancelous bone, via a bore drilled in the bone, anchoring is achieved by applying a compressive force thereon by rotating the central bolt, and thereby generating compressive forces on the elastic material of the buttressing mechanism against the apparatus head (e.g., cover 22c in FIG. 1A). The elastic material of the buttressing mechanism is elastically deformed and thus it is shortened in length while increasing its diameter altogether orthogonal to the longitudinal axis of the apparatus (e.g. using the same principle as compressing a ball, it forms a discus with bigger diameter in comparison to the initial uncompressed ball). The increased diameter of the elastic material of the buttressing mechanism serves to anchor the distal part of the device. When the central bolt 58 is rotated in a first direction e.g., counterclockwise, the compressive forces acting on the elastic material diminish, the elastic material returns to its initial state, pre-pressed form thus allowing for the safe extraction of the apparatus.

In another specific embodiment of the present invention, the distal buttressing mechanism of the fixation device is designed to include an inflatable (like a balloon) capsule. The capsule is made from a type of approved long term implantable material, as indicated by the FDA, for example, Silicon, Rubber, or Nitinol. When the central bolt 58 is rotated, it compress a piston into a chamber (like a syringe), this chamber is filled with isotonic fluid (e.g. normal saline), when the volume of the chamber is decreased (by the action of the piston) the fluid, via pre made bores, is passed into the balloon like capsule thereby inflating the capsule (much like a balloon). The diameter of the capsule increases and by that it allows for the anchoring of the apparatus. When the central bolt rotates in the other direction e.g., counterclockwise, the action of the piston on the fluid chamber is reversed, pressure decreases and the capsule deflates, thus allowing for the safe retrieval of the apparatus.

Bone Fixation Procedure

In a typical bone fixation procedure, after the fractured bone is aligned/reduced to its adequate position, a fixation procedure will be carried out for securing the segments of the fractured bone firmly, close enough to each other as possible. This rigid holding of the bone fragments allows for primary bone healing. Primary bone healing, is a form of intra-membranous healing, which allows for bone synthesis (new bone formation between fragments of bone) to be formed without the formation of callus tissue (i.e., woven, disorganized bone), because callus tissue is disorganized if it forms around joint articulation it can severely restrict their movement, cause accelerated wearing of the joint and cause severe pain to the patient.

The primary bone healing, without callus formation, can only be archived if after the fixation the adjacent parts of the fractured bone are firmly held, the amount of displacement which is allowed between the bone fragments is less than 3% of the bone diameter. If the fragments, after fixation, will be loosely held (more than 3% displacement) a callus formation will ensue. If the displacement is more than 10% of bone diameter, a fibrotic tissue will form (no bone formation) and no bone healing is considered.

The fixation device of the invention is used as follows:

1. The surgeon drills a standard bore in the bone. Either a through bore, or a bore that crosses through the near cortex only. The bore can be drilled in various angels, as indicated by the surgeon needs, according to the type and nature of the fracture. The standard bore sizes are usually 3 to 5 mm.

2. The surgeon inserts the fixation device of the invention into the drilled bore, for example, by using the tooling device. As mentioned hereinabove, the length of the cylindrical housing is shorter than the diameter of the bone if bolt is used, or longer than the diameter of the bone if a nut is used.

3. The surgeon deploys the far buttressing mechanism. The deployment of the buttressing mechanism can be:
(i) Beyond the far cortex, outside the bone, or
(ii) Beyond the near cortex, inside the bone, or
(iii) Inside a pre-drilled bore in a cancelous bone.

The far buttressing mechanism is deployed by counterclockwise rotation of the central bolt (58b). The central bolt is rotated by a rod (23t), the rode transverse the entire length of the tooling device till the base of the buttressing mechanism where it is attached, the rode is rotated by the tooling device, the surgeon activates the tooling device rod by 180° rotation of the tooling device side handle (51x).

4. The surgeon presets the torque of the tooling device created by rotating the torque meter gauge (51Y).

5. The surgeon pulls back the tooling device proximally, thereby pulling the fixation device and presses its buttressing mechanism against the bone (no need for this action in cancelous bone).

6. The surgeon rotate clockwise the tooling device (like an electrical screw driver), while maintaining the pulling pressure, this action locks the near bolt/nut into the cylindrical housing and by is fasten and secures the device, thus forms cleaving forces between the far buttressing mechanism and the near bolt/nut. The amount of cleaving forces is limited by a clutch limiter mechanism (not shown) optionally provided in the tooling device. The clutch limiter mechanism is easily tuned to limit the maximum cleaving forced imposed on the bone.

7. If a nut is used as a proximal fastener (57), the proximal portion of cylindrical housing should be trimmed (the portion of the cylindrical housing protruding toward the surgeon from the proximal side of the bone) this is done by a forcing cone. The cone has a central hole that goes from the cones base to its tip; through this hole the central rod (23t) is passed.

When the cone is forced axially, on the rod, toward the near nut, its tip enters the cylindrical housing thus forcing it to shear over the near nut. This trimming of the cylindrical housing is facilitated by means of pre-made grooves 55q (shown in FIGS. 4H and 8A) running along, in parallel to the axis of, the cylindrical housing.

8. By pressing on the tooling device release mechanism (51z) the tooling device is released from the fixation apparatus (27). The central rod (23t) is preferably a part of the tooling device, the gearing of the central rod to the far buttressing mechanism is achieved by inserting its hexagonal shaped tip to the right-sized hexagonal shaped central bolt head (58b) thus the rod is freely detachable from the fixation apparatus.

9. In order to extract/retrieve/pullout the fixation device:
(i) the tooling device is attached to the fixation apparatus by its near bolts head/nut (57, 24).
(ii) the tooling device is rotated counterclockwise, in order to open the proximal nut (24) and release the cleaving forces applied by the fixation device.
(iii) the buttressing winged mechanism is folded/retrieved. The folding of the wings is achieved by rotating the centrally placed rod (23t) clockwise, this is done by rotating the side handle of the tooling device (51x) 180 degrees.
(iv) the tooling device is pulled out into which the fixation device is held.

10. the fixation device may be used with the gripping element of the invention as follows:
(i) Carry out the steps indicated above for: drilling, insertion of the fixating device, deploying the wings.
(ii) Through another bore 10 to 30 mm apart or on the other side of the fracture (e.g. the adjacent hole in the fixating plate) insert the gripping element. The insertion of the gripping element is done with the same tooling device used to insert the fixation device as hereinabove mentioned. The gripping element is inserted till its side opening:
   1. Protrudes above the far cortex, outside the bone, or
   2. Protrudes above the near cortex, inside the bone, or
   3. Inserted into bore, in case of cancelous bone.
      The gripping element is positioned in a way to allow the side opening to face the fixation device wing.
(iii) By clockwise rotating the central bolt 98 provided in the gripping element, the distal tip of the central bolt is pressed and firmly grips the distal end portion of the anchoring means received in its side opening. Thus, firmly connecting the fixation device to the gripping element.

The rotation of the central bolt 98 is carried out in the same manner mentioned hereinabove.
(iv) The steps of: fastening the proximal bolt/nut and dismounting the tooling device are the same as mentioned hereinabove.
(v) After both the fixation device and gripping element are connected to each other and firmly secured to the bone, the central bolt (58) is clockwise rotated in the fixation device, this rotation pulls the winged buttressing mechanism, such that the pulling forces (when the wing is attached to the gripping element) applies tension between the fixation device and the gripping element. This tension, if applied above a fracture line, will further compress the fracture fragments, thus allowing for a primary healing and reduce the incidence of atrophic non union of the fractured bone.
(vi) Extraction/retrieval of the fixation device and gripping element are the same as mentioned hereinabove:
   Release the grip applied by the gripping element;
   Release the near nut/bolt of both the devices;
   Fold the winged buttressing mechanism of the fixation device;
   Remove the fixation device and the gripping element from their bores with the help of the tooling device.

As described hereinabove, the fixation device establishes a far anchoring by means of winged/discus forming/balloon shaped buttressing mechanism. The buttressing mechanism anchors into/onto the bone. The anchoring is achieved by occupying large surface area on/in the bone. Thus reduce the load per unit area there over. Securing the nut over the proximal side of the bone creates a cleaving (i.e., far and proximal anchors are established) effect on the bone. In turn, the bone fractures are tightened to a plate or to each other.

Reduced bone stress can bring benefits in the following manners: (a) the use of fewer anchors than screws to fixate the same fracture, (b) the probability of fixation failure due to poor bone quality is decrease, and (c) the probability of hardware failure is also decrease.

The aforementioned benefits are further enhanced if the broken bone is of low density bone, i.e., osteoporotic bone.

It should be understood that in a Uni-cortical bone fixation procedure the similar procedure as described hereinabove is carried out, with the exception that the bore is drilled through the near cortex only, the buttressing winged mechanism is deployed inside the bone, and the wings lay on the near cortex from inside.

In the case of a Cancelous-bone fixation, the same procedure as described hereinabove is carried out, with the exception that the bore is drilled through the near cortex into the cancelous bone but not through the far cortex. The winged buttressing mechanism is deployed to anchor into the cancelous bone material, if the anchoring power, of the deployed wings, into the cancelous bone is not strong to establish firm anchoring, one can stock several winged buttressing mechanism upon each other into a fitting cylindrical housing, thus allowing for the deployment of several wings, in different locations, on the longitudinal axis of the cylindrical housing. The deployment of several buttressing wings will allow for larger surface area to contact the cancelous bone, by that to share the cleaving forces onto a larger surface area, decreasing the load from the cancelous bone. It will allow for the fixation device fast anchoring into the cancelous bone.

The fixation device of the invention may be designed such that the curved passage and lateral apertures through which the anchoring means are deployed (22c, 55a) are configured to determine the shape of the deployed anchoring means i.e., to provide near curling as exemplified in FIGS. 7B and 9A.

It should be appreciated that the fixation device and procedure of the present invention employing a gripping element enable using 2 fixation devices in each side of the fracture instead of 3 to 4 screws used today thus reducing to minimum the invasive procedure, the surgical cut, the probability of being infected, shortening healing time and, the level of comfort to the patient.

Improved bone healing may be achieved by injecting bone morphogenic proteins (BMP), growth factors and antibiotics into the hollow cylindrical housing of the fixation device after it is positioned and secured to its final position.

The tooling device of the invention allows the surgeon to carry out the following functions:
(i) Securely gripping of the fixation device.
(ii) Insertion of the fixation device through the pre-drilled bore.
(iii) Deployment of buttressing mechanism by counterclockwise/clockwise rotating an axially located rod, or
(iv) Retrieval of buttressing mechanism by counterclockwise/clockwise rotating an axially located rod, or
(v) Tensioning of the buttressing winged mechanism after being geared to a collector anchor, or
(vi) Insertion of a collector anchor.
(vii) Fastening of the cleaving nut/bolt up to a pre-specified torque to achieve required cleaving force.
(viii) Trimming shaft excess off, ensuring minimal protrusion into surrounding tissue.

As discussed hereinabove in detail, the invention provides an easy to use fixation device that offers the following advantages:
A much stronger fixation:
   (i) Of bone fractured parts to each other, or
   (ii) Of commonly used Hardware (e.g. plates) to the bone, or
   (iii) Of commonly used hardware to the fixation device near end (e.g. cerclage wire, spinal rods, wires to fasten ligament to bone).
   The compression forces that can be generated by the cleaving mechanism, explained hereinabove, are significantly greater than that of conventional screw used today.
   Due to the increased diameter of the far buttressing mechanism when deployed, the increase in compressive forces are distributed over a larger contact area between the far buttressing mechanism and the bone, by distributing the compressive force over a larger area, the load on the bone per unit area is decreased thus allowing for fasten rigid fixation without damaging the bone.
The need to re-operate a patient because of a loose hardware (hardware failure) is decreased.
The commonly used screws as used nowadays, functions by generating compressive forces between the screws head and the bone far cortex. Into the far cortex the screw tip is anchored.
With age, bone mass (density) is decreased; the cortical bone into which the screw is fastened becomes thinner. The thin cortex holds less of the screws threading. The effective surface area between the screw thread and the bone decreases. This decreased surface area for the same compressive forces done by the screw puts greater load on the bone. The bone cannot stand the increased load and micro fracture ensues. This micro fracture is seen as screw “cut out” and hardware failure.
Better healing of the fracture site.
The bone fixation device of the invention, due to its increased rigid fastenings, allows for a primary (intra-membranous) healing to take place. Primary healing occurs if, and only if, minimal displacement of the fractured bone parts is achieved.
Primary healing forms no callus (secondary healing) that might endanger adjacent joint and restrict their movement thus, the invention can be used in fractures transverse into the joint in order to create inter-fragmentary compression.
The time duration for operating a patient is decreased. Nowadays, the common method, to fixate a fracture with plate is to drill 3 to 4 bores on each side of the fracture line. The improved fixation of the invention allows for fewer bores to be drilled in order to archive required fasten secure holding (the number of bores will be reduced to the minimum of 2) thus, allowing for a minimally invasive operating technique.
The costs of operation to the hospital and patient are reduced.
Because of the secure holding and the tooling device, the surgeon operating time will be decreased.
This will save operating room time, overhead time (e.g. time of nurse's, anesthesiologist), time the patient is under general anesthesia and hospitalization time.
Decrease wound infections.
Smaller skin incision and shorter operating time will allow for less surgical site wound infection probability.
The number of actions (steps) performed by the surgeon to fixate the bone during each operation significantly decreases. (e.g. no need to measure the diameter of the bone as done today when inserting screw, the invention has “one size fits all” concept).
Less exposure to ionizing radiation of the patient and surgeon.
Nowadays, in order to ensure that the screw's tip is located in optimal position (reached the far cortex, not protruding into the soft tissue beyond the bone or not reaching the far cortex at all) image intensifier device is used during the operation.
The bone fixation device of the invention, by its buttressing mechanism, can be placed in optimal position without the need for an image intensifier device thus allowing for less exposure to ionizing radiation.
The hardware inventory needed to be kept in the hospital is significantly decreased.
Nowadays, commonly used screw's shaft are stored in variety of sizes in 2 mm steps since different sizes of screws to fit to different bone diameter are required.
The “one size fits all” concept of the bone fixation device of the invention solves this huge inventory stocking.
When a set of screws is opened and only few devices are used, the residual has to be re-sterilized. The need to re-sterilize unused hardware, as for example different sizes of screws, for each operation is eliminated.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different shafts, rods, housings, and other members, described hereinabove may be constructed in different shapes (e.g. having oval, square etc. form in plan view), type of materials, and sizes, differing from those exemplified in the preceding description.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1-19. (canceled)

20. An anchoring device, comprising:
a cylindrical housing having a longitudinal axis;
at least one support pin placed in said cylindrical housing; and
a rod configured for being mechanically linked to said at least one support pin in said cylindrical housing;
wherein said mechanically linkage allow said rod to to manipulate an axial extraction of said at least one support pin, from a distal end of said cylindrical housing, substantially along a perpendicular to said longitudinal axis.

21. The anchoring device of claim 20, wherein said cylindrical housing comprises an internal thread along at least a portion of its inner surface, said rod being threaded over said internal thread.

22. The anchoring device of claim 21, wherein said at least one supporting pin is capable of being at least one of extended from said distal end and retracted to said distal end by means of rotating said rod over said internal thread.

23. The anchoring device of claim 20, wherein said cylindrical housing is sized and shaped to traverse a bone orthogonally to its longitudinal axis.

24. The anchoring device of claim 20, wherein said distal end having at least one lateral aperture sized and shaped to confine the angle of said axial extraction in relation to said cylindrical housing.

25. The anchoring device of claim 20, further comprising at least one gripping element capable of receiving and clamping said at least one support pin along said perpendicular.

26. The anchoring device of claim 25, wherein each said at least one gripping element distal comprising a gripping cylindrical housing, at least one gripping end placed in said cylindrical housing, a gripping rod threaded in said gripping cylindrical housing and mechanically linked to said at least one gripping end, said gripping rod being configured for moving along said gripping cylindrical housing to operate said at least one gripping end for clamping said at least one support pin.

27. The anchoring device of claim 20, wherein said at least one support pin is extended by a compressible member capable of changing its diameter in response to compressive forces applied by said rod.

28. The anchoring device of claim 20, wherein said at least one support pin is extended by an inflatable member capable of changing its diameter responsive to inflation media flown thereinto.

29. The anchoring device of claim 20, wherein at least one of said at least one support pin having a blunt end.

30. The anchoring device of claim 20, wherein the length of said cylindrical housing is less than 60 millimeters.

31. An anchoring device, comprising:
a cylindrical housing having a longitudinal axis and distal and proximal ends;
at least one support pin placed in said cylindrical housing;
a rod configured for being mechanically linked to said at least one support pin in said cylindrical housing;
a fixation plate configured for being tightened to said proximal end substantially in perpendicular to said longitudinal axis;
wherein said rod is configured for moving along said longitudinal axis to induce and extraction of said at least one support pin from said cylindrical housing via said distal end.

32. The anchoring device of claim 31, wherein said proximal end having an external thread on at least a portion of the external surface thereof, said fixation plate is tightened between said proximal end and a nut which is threaded over said external thread.

33. The anchoring device of claim 31, wherein said distal end comprises at least one lateral aperture configured to confine said at least one support pin, during said extraction, to a predetermined angle relative to said longitudinal axis.

34. The anchoring device of claim 31, further comprising at least one gripping element each comprising a gripping cylindrical housing, at least one gripping end placed in said cylindrical housing, a gripping rod threaded in said gripping cylindrical housing and mechanically linked to said at least one gripping end, said gripping rod being configured for moving along said gripping cylindrical housing to operate said at least one gripping end for clamping said at least one support pin, said fixation plate configured for being tightened to said at least one gripping element.

35. The anchoring device of claim 31, further comprising at least one additional cylindrical housing each having at least one additional support pin and an additional rod placed therein; wherein said fixation plate configured for being tightened to a proximal end of each said at least one additional cylindrical housing.

36. A method for bone fixation, comprising:
providing a bone anchor having a cylindrical housing encircling at least one supporting pin and a rod being mechanically linked thereto;
drilling a bore in a bone to be fixated between a proximal cortex and a distal cortex;
inserting said bone anchor into said bore so that said bone anchor simultaneously traversing said distal and proximal cortexes; and
manipulating the rod to induce an extraction of said at least one support pin from said cylindrical housing via said distal cortex, externally to said bone.

37. The method of claim 36, further comprising tightening a fixation plate to said proximal end and to an external surface of said bone in proximity to said proximal end.

38. The method of claim 37, further comprising drilling at least one additional bore in said bone, substantially parallel to said bore and inserting each said at least one additional bore with at least one of an additional bone anchor and a gripping element and fixating said at least one of said additional bone anchor and said gripping element to said fixation plate for enhancing a mechanical bone fixation performed by said bone anchor.

39. The method of claim 36, wherein said manipulating comprises using an external tool for rotating said rod along a thread on an inner surface of said cylindrical housing.

40. The method of claim 36, further comprising threading a nut to said proximal end so that said bone anchor being secured to said bone.

41. A method for bone fixation, comprising:
providing a bone anchor having a cylindrical housing encircling at least one supporting pin and a rod being mechanically linked thereto;
drilling a bore in a bone to be fixated between a proximal cortex and a cancelous bone segment;
inserting said bone anchor into said bore so that said bone anchor simultaneously traversing said proximal cortex and said cancelous bone segment; and
manipulating the rod to induce an extraction of said at least one support pin from said cylindrical housing via said proximal cortex, in said cancelous bone segment.
